(19) United States
(12) Patent Application Publication
(10) Pub. No.: US 2017/0049811 A1
(43) Pub. Date: Feb. 23, 2017

(54) MODIFIED SINGLE DOSE, MICROPLEGIC APPROACH TO CARDIOPLEGIA FOR ADULT HEART

(71) Applicants: Catherine E. Berry, Atlanta, GA (US); Candice Kalin, Atlanta, GA (US)

(72) Inventors: Catherine E. Berry, Atlanta, GA (US); Candice Kalin, Atlanta, GA (US)

(21) Appl. No.: 15/308,269

(22) PCT Filed: May 1, 2015
(86) PCT No.: PCT/US15/28777

§ 371 Date: Nov. 1, 2016

Related U.S. Application Data
(60) Provisional application No. 61/987,083, filed on May 1, 2014.
(60) Provisional application No. 61/994,357, filed on May 16, 2014.

(51) Int. Cl. A61K 33/06; A61K 31/216; A61K 31/047; A61K 9/08; A61K 33/00; A61K 31/167

(52) U.S. Cl. CPC A61K 33/06; A61K 33/00; A61K 31/167; A61K 31/047; A61K 9/08; A61K 31/216

(57) ABSTRACT

Disclosed are compositions and methods for providing a modified single dose all blood approach to cardioplegia and reanimation. The disclosed method of preserving a heart comprises of administering a cardiac arrest induction solution to the blood of a subject, perfusing the heart with the blood containing the cardiac arrest induction solution, and reanimating the heart with a cardiac reanimation solution. The cardiac arrest induction solution disclosed herein consists essentially of Potassium, a calcium channel blocker, a sodium channel blocker, and a free radical scavenger, while the cardiac reanimation solution comprises of calcium channel blocker and one or more of a sodium channel blocker, a beta receptor blocker, potassium, and a buffer.

### I. BACKGROUND

Since the introduction of elective cardiac arrest in the 1950's, numerous cardioprotective strategies have been used to preserve heart function during an ischemic state. Traditionally, cardioplegia has used a potassium-induced electromechanical arrest with an initial induction dose, followed by repeated maintenance doses, approximately every twenty minutes. Crystalloid or partial blood based carriers have been used, with varying ratios of crystalloid to blood. Most solutions are hyperkalemic and extracellular in nature, however intracellular-type, crystalloid based solutions are available.

The traditional cardioplegia method requires intermittent maintenance dosing, be it every 15 to 30 minutes, which cause sporadic, frequent interruptions to the surgeon and the surgical team during the procedure. The Quest myocardial protection system was developed in the late 90's and the era of “microplegia” was born. In addition to increasing myocardial edema, crystalloid cardioplegia solutions have long been recognized as causing increased hemodilution, thereby contributing to intraoperative blood transfusion requirements. This delivery system provided a way to deliver micro amounts of chemical agents in an all blood carrier, thus limiting hemodilution, reducing myocardial edema, and providing the added oxygen carrying capacity of a self-buffering, blood rich environment. However, intermittent maintenance dosing was still required.

The cardioplegia solution, developed for pediatric use by Dr. del Nido in the 90's, provides a single dosing strategy, but continues to utilize a crystalloid based carrier in a four parts crystalloid to one part blood formulation. While widely accepted in pediatrics, it has been gaining popularity in the adult arena due to the ability to see arrest times of greater than 60 to 120 minutes with one single dose rather than the traditional repeated maintenance doses. However, the method utilizes crystalloid based carriers and therefore suffers from all of the deficiencies associated with such a methodology.

Despite the presence of a single dose cardioplegia method, there remains a real need and potential benefit from developing a new cardioplegia methods and compositions that retains the high oxygen carrying capacity, low hemodilution, and reduced myocardial edema qualities of an all blood, microplegia while also providing a lower maintenance modified single-dosing, sustained arrest, “del Nido” type chemical component, and terminal warm reanimation strategy.

### II. SUMMARY

Disclosed are methods and compositions related to a modified cardioprotective strategy to accommodate changes in the aging adult heart, while utilizing the benefits of microplegia delivery and sustained arrest single dosing cardioplegia.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 illustrates a retrospective look at the longest period in minutes between doses.

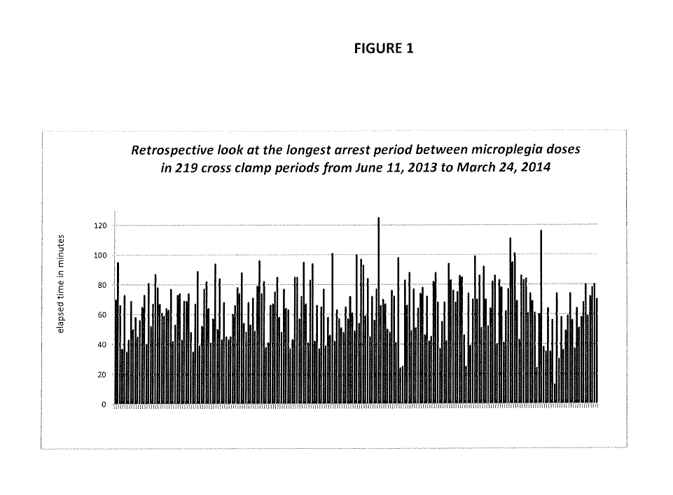

FIG. 2 shows a comparison of the average amount of time in minutes for the longest arrest period (lap) between doses, along with the minimum, maximum and mode lap in a retrospective review of 219 procedures alongside the cardiac surgery “gold standard” of 20 minutes.

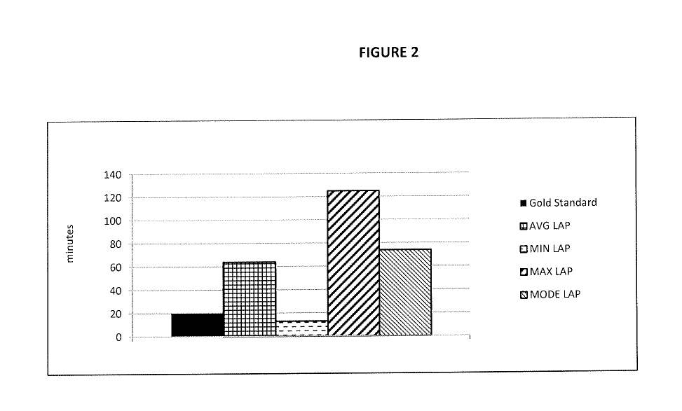

FIG. 3 shows a flow chart demonstrating the process.

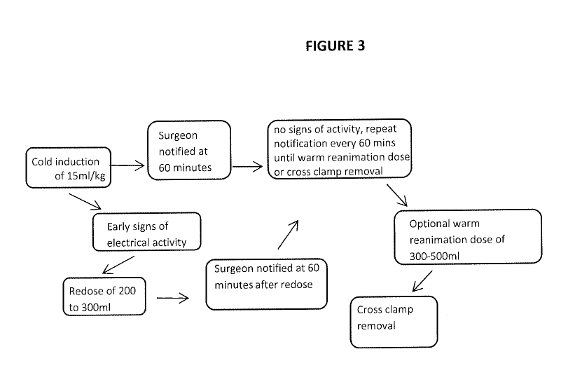

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular cardiac arrest induction solution or cardiac reanimation solution is disclosed and discussed and a number of modifications that can be made to the compositions including the inclusion of Potassium and/or a buffer such as Sodium Bicarbonate are discussed, specifically contemplated is each and every combination and permutation of the cardiac arrest induction solution or cardiac reanimation solution and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Traditionally, cardioplegia is the elective arrest of the heart using a 1:4 solution of blood and crystalloid with lidocaine, magnesium, and potassium during on pump open heart surgeries. Elective cardiac arrest was introduced in 1955, by rapidly injecting into the aortic root, after aortic cross-clamping, a 2.5% potassium citrate solution in warm blood to arrest the heart. It is well known by those skilled in the art to employ during transplantation of an organ and cardioplegia during cardiac surgery a solution that will preserve the organ during the complete interruption of its blood and oxygen supply. Generally, these solutions rely on substantially arresting the organ's metabolism with the use of chemicals and by employing temperatures of 4.degree. Centigrade and lower. This substantial slowing of the metabolism of the organ permits the energy stores in the organ at the time of harvesting to be consumed at a slower rate. Accordingly, the preservation time of these solutions is limited by the available energy stores present at the time of harvesting and the rate of their consumption. Organ preservation such as for example, preservation of the heart, is limited by the fact that currently available solutions are able only to safely preserve organs for very limited periods of time. For the heart, this time is generally less than six hours. This relatively short period of time severely limits the distance that these organs may be transported and imposes great demands upon the surgeon to complete the complex and delicate transplant surgery in an expeditious manner. Another disadvantage of these solutions is that they do not preserve the organ by promoting anaerobic glycolysis. For example, it is well known by those skilled in the art that prolonged myocardial preservation is limited by the heart's inability to maintain high energy stores and low intracellular calcium levels during ischemia. Anaerobic glycolysis, which is the only potential source of adenosine 5′-triphosphate (ATP) during ischemia, is inhibited by the accumulation of lactate and in the myocytes. These waste products inhibit energy production by the organ while the organ is outside of the patient's body. As a result, ATP production during ischemia is inhibited. It is known by those skilled in the art that low ATP levels are associated with detrimental morphologic changes in the heart.

### Cardiac Arrest Induction Solution

In one aspect, disclosed herein is a cardiac arrest induction solution. Typically, cardiac arrest induction solutions comprise several components that provide benefits to the recipient. For example, the cardiac arrest induction solution can comprise a Calcium channel blocker, a sodium channel blocker, and a free radical scavenger. Cardiac arrest induction solutions can also comprise a source of potassium and a buffer. In one aspect, disclosed herein are cardiac arrest induction solutions consisting essentially of Potassium, a calcium channel blocker, a sodium channel blocker, and a free radical scavenger.

In one aspect, the disclosed cardiac arrest induction solution comprises a calcium channel blocker. It is understood and herein contemplated that any calcium channel blocker can be used for this purpose. For example, the calcium channel blocker can be a source of Magnesium such as, Magnesium sulfate. Other examples of calcium channel blockers that can be used with the disclosed invention, include but are not limited to chelated magnesium, magnesium aspartate, magnesium carbonate, magnesium chloride, magnesium citrate, magnesium diglycine, magnesium dihydrogen diphosphate, magnesium disuccinate hydrate, magnesium gluconate, magnesium glycerophosphate, magnesium glycinate, magnesium hydroxide, magnesium lactate, magnesium malate, magnesium murakab, magnesium orotate, magnesium oxide, magnesium phosphate, magnesium pidolate, and magnesium salicylate. In one aspect, disclosed herein is a cardiac arrest induction solution comprising a calcium channel blocker wherein the calcium channel blocker is Magnesium sulfate.

It is understood and herein contemplated that the cardiac arrest induction solution can comprise any amount of calcium channel blocker suitable for said purpose of inducing cardiac arrest. For example, the cardiac arrest induction solution can comprise 0.1 to 2 g of a calcium channel blocker (such as, for example, Magnesium in the form of Magnesium sulfate) per liter of blood cardioplegia delivered. In one aspect, disclosed herein are cardiac arrest induction solutions wherein 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 g or more of a calcium channel blocker, such as Magnesium sulfate are provided per liter of blood cardioplegia delivered. For example, the cardiac arrest induction solution can comprise from about 0.1 to about 2.0 g of a calcium channel blocker, such as Magnesium, per liter of blood cardioplegia delivered; from about 0.5 to about 2.0 g of a calcium channel blocker, such as Magnesium, per liter of blood cardioplegia delivered; from about 0.8 to about 2.0 g of a calcium channel blocker, such as Magnesium, per liter of blood cardioplegia delivered; from about 1.0 to about 2.0 g of a calcium channel blocker, such as Magnesium, per liter of blood cardioplegia delivered; from about 1.2 to about 1.8 g of a calcium channel blocker, such as Magnesium, per liter of blood cardioplegia delivered; from about 1.5 to about 1.7 g of a calcium channel blocker, such as Magnesium, per liter of blood cardioplegia delivered; or about 1.6 g of a calcium channel blocker, such as Magnesium, per liter of blood cardioplegia delivered.

The disclosed cardiac arrest induction solutions can also comprise a sodium channel blocker, such as, for example, Lidocaine. It is understood and herein contemplated that other sodium channel blockers that can be used include any class 1 antiarrythmic, including but not limited to xylocaine, lignocaine, procainamide, quinidine, and disopyramide. Additionally, procaine can also be used as a sodium channel blocker. In one aspect, disclosed herein is a cardiac arrest induction solution comprising a sodium channel blocker wherein the sodium channel blocker is Lidocaine.

It is understood and herein contemplated that the cardiac arrest induction solution can comprise any amount of sodium channel blocker suitable for said purpose of inducing cardiac arrest. For example, the cardiac arrest induction solution can comprise 0.1 to 200 mg of a sodium channel blocker (such as, for example, Lidocaine) per liter of blood cardioplegia delivered. In one aspect, disclosed herein are cardiac arrest induction solutions wherein 0.1, 0.2, 0.3, 0.4, 0.5, 0.6, 0.7, 0.8, 0.9, 1.0, 1.1, 1.2, 1.3, 1.4, 1.5, 2.0, 2.5, 3.0, 4.0, 4.5, 5.0, 5.5, 6.0, 6.5, 7.0, 7.5, 8.0, 8.5, 9.0, 9.5, 10, 11, 12, 12, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200 mg or more of a sodium channel blocker, such as Lidocaine, per liter of blood cardioplegia delivered are provided. For example, the cardiac arrest induction solution can comprise from about 0.1 mg to about 200 mg of a sodium channel blocker, such as Lidocaine, per liter of blood cardioplegia delivered; from about 10 mg to about 200 mg of a sodium channel blocker, such as Lidocaine, per liter of blood cardioplegia delivered; from about 50 mg to about 150 mg of a sodium channel blocker, such as Lidocaine, per liter of blood cardioplegia delivered; from about 80 mg to about 120 mg of a sodium channel blocker, such as Lidocaine, per liter of blood cardioplegia delivered; from about 90 mg to about 110 mg of a sodium channel blocker, such as Lidocaine, per liter of blood cardioplegia delivered; from about 100 mg to about 105 mg of a sodium channel blocker, such as Lidocaine, per liter of blood cardioplegia delivered; or about 104 mg of a sodium channel blocker, such as Lidocaine, per liter of blood cardioplegia delivered.

In one aspect, the disclosed cardiac arrest induction solution can comprise a free radical scavenger. It is understood and contemplated herein that the free radical scavenger can also be an osmotic diuretic. For example, it is contemplated herein that the free radical scavenger can be Mannitol, N-acetylcycteine, or any hypertonic saline substitute. Accordingly, in one aspect disclosed herein is a cardiac arrest induction solution comprising a free radical scavenger, wherein the free radical scavenger is Mannitol.

It is understood and herein contemplated that the cardiac arrest induction solution can comprise any amount of free radical scavenger suitable for said purpose of inducing cardiac arrest. For example, the cardiac arrest induction solution can comprise 0.1 to 2 g of free radical scavenger (such as, for example, Mannitol). In one aspect, disclosed herein are cardiac arrest induction solutions wherein 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 g or more of a free radical scavenger, such as Mannitol per liter of blood cardioplagia delivered are provided. For example, the cardiac arrest induction solution can comprise from about 0.1 to about 5.0 g of a free radical scavenger, such as Mannitol, per liter of blood cardioplegia delivered; from about 0.5 to about 4.5 g of a free radical scavenger, such as Mannitol, per liter of blood cardioplegia delivered; from about 1.0 to about 4.0 g of a free radical scavenger, such as Mannitol, per liter of blood cardioplegia delivered; from about 1.5 to about 3.0 g of a free radical scavenger, such as Mannitol, per liter of blood cardioplegia delivered; from about 2.0 to about 3.0 g of a free radical scavenger, such as Mannitol, per liter of blood cardioplegia delivered; from about 2.2 to about 3.0 g of a free radical scavenger, such as Mannitol, per liter of blood cardioplegia delivered; from about 2.4 to about 2.8 g of a free radical scavenger, such as Mannitol, per liter of blood cardioplegia delivered; or about 2.6 g of a free radical scavenger, such as Mannitol, per liter of blood cardioplegia delivered.

In one aspect, disclosed herein is a cardiac arrest induction solution comprising Lidocaine, Mannitol, and Magnesium (for example Magnesium sulfate).

In addition to the disclosed sodium channel blocker, free radical scavenger, and calcium channel blocker it is contemplated herein that the disclosed cardiac arrest induction solution can optionally comprise Potassium. It is understood and herein contemplated that Potassium can be provided as a component in a cardiac arrest induction solution, as a separate component that is mixed with a cardiac arrest induction solution prior to administration or provided concurrently with a cardiac arrest induction solution.

It is understood and herein contemplated that the cardiac arrest induction solution can comprise any amount of Potassium suitable for said purpose of inducing cardiac arrest. For example, the cardiac arrest induction solution can comprise 0.1 to 2 g of Potassium. In one aspect, disclosed herein are cardiac arrest induction solutions wherein 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 6, 7, 8, 9, 10, 11, 12, 13, 14, 15, 16, 17, 18, 19, 20, 21, 22, 23, 24, 25 mmol or more of Potassium per liter of blood cardioplagia delivered are provided. It is understood that as disclosed herein, 1 mmol per liter of blood cardioplegia delivered is equal to 1 milliequivalent. Therefore, where a particular amount of Potassium is provided, such as, for example, 22 milliequivalents, this is equal to 22 mmol of Potassium per liter of blood cardioplegia delivered. For example, the cardiac arrest induction solution can comprise from about 0.1 to about 25 mmol of Potassium per liter of blood cardioplegia delivered; from about 1.0 to about 25 mmol of Potassium per liter of blood cardioplegia delivered; from about 5.0 to about 25 mmol of Potassium per liter of blood cardioplegia delivered; from about 10 to about 25 mmol of Potassium per liter of blood cardioplegia delivered; from about 15 to about 25 mmol of Potassium per liter of blood cardioplegia delivered; from about 20 to about 25 mmol of Potassium per liter of blood cardioplegia delivered; from about 21 to about 23 mmol of Potassium per liter of blood cardioplegia delivered; or about 22 mmol of Potassium per liter of blood cardioplegia delivered.

In one aspect, disclosed herein is a cardiac arrest induction solution comprising Potassium, Lidocaine, Magnesium, and Mannitol. It is understood and herein contemplated that situations can arise where further active ingredients in the cardiac arrest solution are not desired. Therefore, in one aspect disclosed herein are cardiac arrest induction solutions consisting essentially of a calcium channel blocker (such as, for example Magnesium including Magnesium in the form of Magnesium sulfate), a sodium channel blocker (such as, for example, Lidocaine), and a free radical scavenger (such as, for example, Mannitol). Also disclosed are cardiac arrest induction solutions consisting essentially of a calcium channel blocker (such as, for example Magnesium including Magnesium in the form of Magnesium sulfate), Potassium, a sodium channel blocker (such as, for example, Lidocaine), and a free radical scavenger (such as, for example, Mannitol).

### Cardiac Reanimation Solution

In another aspect, disclosed herein is a cardiac reanimation solution comprising a calcium channel blocker and one or more of a sodium channel blocker, a beta receptor blocker, potassium, and a buffer. For example, disclosed herein are cardiac reanimation solutions comprising a calcium channel blocker, a sodium channel blocker, and potassium. Also disclosed are cardiac reanimation solutions comprising a calcium channel blocker, a beta receptor blocker, and potassium. Also, disclosed herein are cardiac reanimation solutions comprising a calcium channel blocker, a beta receptor blocker, and a sodium channel blocker. Also disclosed herein are cardiac reanimation solutions comprising a calcium channel blocker, a beta receptor blocker, potassium, and a sodium channel blocker.

It is understood and herein contemplated that any calcium channel blocker can be used for this purpose. For example, the calcium channel blocker can be a source of Magnesium such as, Magnesium sulfate. Other examples of calcium channel blockers that can be used with the disclosed invention, include but are not limited to chelated magnesium, magnesium aspartate, magnesium carbonate, magnesium chloride, magnesium citrate, magnesium diglycine, magnesium dihydrogen diphosphate, magnesium disuccinate hydrate, magnesium gluconate, magnesium glycerophosphate, magnesium glycinate, magnesium hydroxide, magnesium lactate, magnesium malate, magnesium murakab, magnesium orotate, magnesium oxide, magnesium phosphate, magnesium pidolate, and magnesium salicylate. In one aspect, disclosed herein is a cardiac reanimation solution comprising a calcium channel blocker wherein the calcium channel blocker is Magnesium sulfate.

It is understood and herein contemplated that the cardiac reanimation solution can comprise any amount of calcium channel blocker suitable for said purpose of restoring cardiac function. For example, the cardiac reanimation solution can comprise 0.1 to 2 g of a calcium channel blocker (such as, for example, Magnesium in the form of Magnesium sulfate) per liter of blood cardioplegia delivered. In one aspect, disclosed herein are cardiac reanimation solutions wherein 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 g or more of a calcium channel blocker, such as Magnesium sulfate are provided per liter of blood cardioplegia delivered. For example, the cardiac reanimation solution can comprise from about 0.1 to about 2.0 g of a calcium channel blocker, such as Magnesium, per liter of blood cardioplegia delivered; from about 0.5 to about 1.5 g of a calcium channel blocker, such as Magnesium, per liter of blood cardioplegia delivered; from about 0.6 to about 1.4 g of a calcium channel blocker, such as Magnesium, per liter of blood cardioplegia delivered; from about 0.7 to about 1.3 g of a calcium channel blocker, such as Magnesium, per liter of blood cardioplegia delivered; from about 0.8 to about 1.2 g of a calcium channel blocker, such as Magnesium, per liter of blood cardioplegia delivered; from about 0.9 to about 1.1 g of a calcium channel blocker, such as Magnesium, per liter of blood cardioplegia delivered; or about 1.0 g of a calcium channel blocker, such as Magnesium, per liter of blood cardioplegia delivered.

The disclosed cardiac reanimation solutions can also comprise a sodium channel blocker, such as, for example, Lidocaine. It is understood and herein contemplated that other sodium channel blockers that can be used include any class 1 antiarrythmic, including but not limited to xylocaine, lignocaine, procainamide, quinidine, and disopyramide. Additionally, procaine can also be used as a sodium channel blocker. In one aspect, disclosed herein is a cardiac reanimation solution comprising a sodium channel blocker wherein the sodium channel blocker is Lidocaine.

It is understood and herein contemplated that the cardiac reanimation solution can comprise any amount of sodium channel blocker suitable for said purpose of restoring cardiac function. For example, the cardiac reanimation solution can comprise 0.1 to 200 mg of a sodium channel blocker (such as, for example, Lidocaine) per liter of blood cardioplegia delivered. In one aspect, disclosed herein are cardiac reanimation solutions wherein 0.1, 0.2, 0.3, 0.4, 0.5, 0.6, 0.7, 0.8, 0.9, 1.0, 1.1, 1.2, 1.3, 1.4, 1.5, 2.0, 2.5, 3.0, 4.0, 4.5, 5.0, 5.5, 6.0, 6.5, 7.0, 7.5, 8.0, 8.5, 9.0, 9.5, 10, 11, 12, 12, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200 mg or more of a sodium channel blocker, such as Lidocaine, per liter of blood cardioplegia delivered are provided. For example, the cardiac reanimation solution can comprise from about 0.1 mg to about 200 mg of a sodium channel blocker, such as Lidocaine, per liter of blood cardioplegia delivered; from about 10 mg to about 200 mg of a sodium channel blocker, such as Lidocaine, per liter of blood cardioplegia delivered; from about 50 mg to about 150 mg of a sodium channel blocker, such as Lidocaine, per liter of blood cardioplegia delivered; from about 80 mg to about 120 mg of a sodium channel blocker, such as Lidocaine, per liter of blood cardioplegia delivered; from about 90 mg to about 110 mg of a sodium channel blocker, such as Lidocaine, per liter of blood cardioplegia delivered; from about 95 mg to about 105 mg of a sodium channel blocker, such as Lidocaine, per liter of blood cardioplegia delivered; or about 100 mg of a sodium channel blocker, such as Lidocaine, per liter of blood cardioplegia delivered.

In another aspect the disclosed cardiac reanimation solutions can further comprise a beta receptor blocker, such as, for example, the cardioselective beta receptor blocker, Esmolol. It is understood and herein contemplated that the beta receptor blocker can be a cardioselective beta receptor blocker. In one aspect, the beta receptor blocker can be comprise any beta receptor blocker including but not limited to cardioselective beta receptor blockers such as, for example, esmolol, esmolol derivatives, atenolol, metapropolol, bisoprolol, betaxolol. acebutolol, and non-selective beta receptor blockers such as, for example, timolol, propranolol, nadolol, and sotalol.

It is understood and herein contemplated that the cardiac reanimation solution can comprise any amount of beta receptor blocker suitable for said purpose of restoring cardiac function. For example, the cardiac reanimation solution can comprise 0.1 to 200 mg of a beta receptor blocker (example, a cardioselective beta receptor blocker, such as esmolol) per liter of blood cardioplegia delivered. In one aspect, disclosed herein are cardiac reanimation solutions wherein 0.1, 0.2, 0.3, 0.4, 0.5, 0.6, 0.7, 0.8, 0.9, 1.0, 1.1, 1.2, 1.3, 1.4, 1.5, 2.0, 2.5, 3.0, 4.0, 4.5, 5.0, 5.5, 6.0, 6.5, 7.0, 7.5, 8.0, 8.5, 9.0, 9.5, 10, 11, 12, 12, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200 mg or more of a beta receptor blocker, such as Esmolol, per liter of blood cardioplegia delivered are provided. For example, the cardiac reanimation solution can comprise from about 0.1 mg to about 200 mg of a beta receptor blocker, such as Esmolol, per liter of blood cardioplegia delivered; from about 10 mg to about 200 mg of a beta receptor blocker, such as Esmolol, per liter of blood cardioplegia delivered; from about 50 mg to about 150 mg of a beta receptor blocker, such as Esmolol, per liter of blood cardioplegia delivered; from about 80 mg to about 120 mg of a beta receptor blocker, such as Esmolol, per liter of blood cardioplegia delivered; from about 90 mg to about 110 mg of a beta receptor blocker, such as Esmolol, per liter of blood cardioplegia delivered; from about 95 mg to about 105 mg of a beta receptor blocker, such as Esmolol, per liter of blood cardioplegia delivered; or about 100 mg of a beta receptor blocker, such as Esmolol, per liter of blood cardioplegia delivered.

In one aspect, disclosed herein are cardiac reanimation solutions comprising Magnesium and Lidocaine. Also disclosed herein are cardiac reanimation solutions comprising Magnesium and Esmolol. Also disclosed herein are cardiac reanimation solutions comprising Magnesium, Esmolol, and Lidocaine. It is contemplated herein that there are instances where limited active ingredients are desired in the cardiac reanimation solution. Accordingly, disclosed herein are cardiac reanimation solution consisting essentially of a calcium channel blocker (such as, for example Magnesium including Magnesium in the form of Magnesium sulfate), a sodium channel blocker (such as, for example, Lidocaine), and beta receptor blocker (such as, for example, the cardioselective beta receptor blocker esmolol). In another aspect, the cardiac reanimation solution consists essentially of a calcium channel blocker (such as, for example Magnesium including Magnesium in the form of Magnesium sulfate), a sodium channel blocker (such as, for example, Lidocaine), sodium bicarbonate, and beta receptor blocker (such as, for example, the cardioselective beta receptor blocker esmolol). Thus, in one aspect, disclosed here are cardiac reanimation solutions consisting essentially of Magnesium, Esmolol, Sodium Bicarbonate, and Lidocaine.

In a further aspect, any of the disclosed cardiac reanimation solutions above can further comprise a free radical scavenger. In aspect where the cardiac reanimation solution further comprises a free radical scavenger it is contemplated herein that the free radical scavenger can be Mannitol, N-acetylcycteine, or any hypertonic saline substitute. Accordingly, in one aspect disclosed herein is a cardiac reanimation solution comprising a free radical scavenger, wherein the free radical scavenger is Mannitol. For example, in one aspect, the cardiac reanimation solution can be comprise calcium channel blocker (such as, for example Magnesium including Magnesium in the form of Magnesium sulfate), a sodium channel blocker (such as, for example, Lidocaine), and a free radical scavenger (such as, for example, Mannitol). Thus, in one aspect, not only can the cardiac reanimation solution comprise the same components as the cardiac arrest indication solution; the cardiac reanimation solution can, in fact, be the same solution as the cardiac arrest induction solution.

It is understood and herein contemplated that the cardiac reanimation solution can comprise any amount of free radical scavenger suitable for said purpose of inducing cardiac arrest. For example, the cardiac reanimation solution can comprise 0.1 to 2 g of free radical scavenger (such as, for example, Mannitol). In one aspect, disclosed herein are cardiac reanimation solutions wherein 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 g or more of a free radical scavenger, such as Mannitol per liter of blood cardioplagia delivered are provided. For example, the cardiac reanimation solution can comprise from about 0.1 to about 5.0 g of a free radical scavenger, such as Mannitol, per liter of blood cardioplegia delivered; from about 0.5 to about 4.5 g of a free radical scavenger, such as Mannitol, per liter of blood cardioplegia delivered; from about 1.0 to about 4.0 g of a free radical scavenger, such as Mannitol, per liter of blood cardioplegia delivered; from about 1.5 to about 3.0 g of a free radical scavenger, such as Mannitol, per liter of blood cardioplegia delivered; from about 2.0 to about 3.0 g of a free radical scavenger, such as Mannitol, per liter of blood cardioplegia delivered; from about 2.2 to about 3.0 g of a free radical scavenger, such as Mannitol, per liter of blood cardioplegia delivered; from about 2.4 to about 2.8 g of a free radical scavenger, such as Mannitol, per liter of blood cardioplegia delivered; or about 2.6 g of a free radical scavenger, such as Mannitol, per liter of blood cardioplegia delivered.

### Pharmaceutical Carriers

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled ill the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers (such as, for example, NaHCO₃), preservatives, surface active agents and the like in addition to the molecule of choice. Pharmaceutical compositions may also include one or more active ingredients such as antimicrobial agents, anti-inflammatory agents, anesthetics, and the like. Thus, for example, disclosed herein are cardiac arrest induction solutions and cardiac reanimation solutions comprising a buffer, such as, for example NaHCO₃. In one aspect, disclosed herein are cardiac reanimation solutions comprising Magnesium, Lidocaine, Esmolol, and Sodium Bicarbonate. Also disclosed herein are cardiac arrest induction solutions comprising Magnesium, Lidocaine, Mannitol, and Sodium bicarbonate. Where a buffer, such as Sodium Bicarbonate is provided, it is understood that the buffer can be provided in solution with the remainder of the components of the cardiac reanimation solution or cardiac arrest induction solution, administered concurrently with the cardiac reanimation solution or cardiac arrest induction solution, or mixed with the remainder of the components of the cardiac reanimation solution or cardiac arrest induction solution prior to administration. It is further contemplated herein that where a buffer such as sodium bicarbonate is provided, the amount of buffer can comprise 0.1 to 25 mmol per liter of blood cardioplegia delivered. For example, the buffer such as, sodium bicarbonate can be provided from about 0.1 mmol to about 25 mmol per liter of blood cardioplegia delivered, from about 1 mmol to about 20 mmol per liter of blood cardioplegia delivered, from about 5 mmol to about 15 mmol per liter of blood cardioplegia delivered, from about 5 mmol to about 10 mmol per liter of blood cardioplegia delivered from about 3 mmol to about 7 mmol per liter of blood cardioplegia delivered, or from about 4 mmol to about 6 mmol per liter of blood cardioplegia delivered. Put another way buffers comprising 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, 16, 17, 18, 19, 20, 21, 22, 23, 24, 25 mmol or more sodium bicarbonate per liter of blood cardioplegia are provided herein. Thus, for example, disclosed herein are cardiac reanimation solutions comprising a sodium channel blocker (such as, for example, Lidocaine), a calcium channel blocker (such as, for example magnesium sulfate), a beta receptor blocker (such as, for example, esmolol), and sodium bicarbonate, wherein the sodium bicarbonate comprises 5 mmol per liter of blood cardioplegia delivered. It is further understood that the mmol values presented herein can also be referred to as milliequivalents where 1 mmol per liter of blood cardioplegia is equal to 1 milliequivalent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Blood is also a suitable carrier. For example, the carrier can be the patient's own blood or blood from a donor. Preservatives and other additives may also be present such as, for example, antimicrobials, anti-oxidants, chelating agents, and inert gases and the like.

It is understood that the disclosed cardiac arrest induction solution and cardiac reanimation solution can be used with a blood carrier and therefore the need for a buffer such as, for example, NaHCO₃ (sodium bicarbonate) would be optional as the blood carrier would provide natural buffering properties. Thus, in one aspect, disclosed herein are cardiac arrest induction solutions wherein the cardiac arrest induction solution does not comprise NaHCO₃. Also, in one aspect, disclosed herein are cardiac reanimation solutions wherein the cardiac reanimation solution does not comprise NaHCO₃. That is, disclosed herein are cardiac arrest induction solutions and cardiac reanimation solutions wherein the carrier solution is a bufferless solution.

### Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include any cardiac arrest solution and/or any cardiac reanimation solution disclosed herein. For example, disclosed is a kit for inducing cardiac arrest and reanimating a heart, comprising a cardiac arrest induction solution comprising a calcium channel blocker (such as, for example, Magnesium), a sodium channel blocker (such as, for example, Lidocaine), and a free radical scavenger (such as, for example, Mannitol) and a cardiac reanimation solution comprising a calcium channel blocker (such as, for example, Magnesium), a sodium channel blocker (such as, for example, Lidocaine), and a beta blocker (such as, for example, Esmolol). Also disclosed is a kit for inducing cardiac arrest and reanimating a heart, comprising a cardiac arrest induction solution comprising a calcium channel blocker (such as, for example, Magnesium), a sodium channel blocker (such as, for example, Lidocaine), and a free radical scavenger (such as, for example, Mannitol) and a cardiac reanimation solution comprising a calcium channel blocker (such as, for example, Magnesium), a sodium channel blocker (such as, for example, Lidocaine), and a free radical scavenger (such as, for example, Mannitol). In one aspect, disclosed are kits wherein the cardiac arrest induction solution and the cardiac reanimation solution comprise the same components or are the same solution. It is understood that the disclosed kits can further comprise Potassium and sodium bicarbonate. The Potassium and Sodium Bicarbonate can be provided as part of or separate from the cardiac arrest induction solution and/or the cardiac reanimation solution respectively.

### C. METHODS OF PRESERVING A HEART

In one aspect, the cardiac arrest induction solutions and cardiac reanimation solutions disclosed herein can be used to preserve cardiac function during an arrested ischemic state such as, for example, during a transplant or cardiac bypass surgery. Thus, disclosed herein are methods of preserving a heart during a surgical procedure comprising administering a cardiac arrest induction solution to the blood of a subject, perfusing the heart with the blood containing the cardiac arrest induction solution, and reanimating the heart with a cardiac reanimation solution.

In another aspect, it is understood and herein contemplated that there can arise occasions where the cardiac reanimation solution is not used in the method. That is, the method of preserving cardiac function comprises administering cardiac arrest induction solution to the subject. Accordingly, disclosed herein, are methods of preserving a heart during a surgical procedure comprising administering a cardiac arrest induction solution to the blood of a subject and perfusing the heart with the blood containing the cardiac arrest induction solution.

It is understood and herein contemplated that the cardiac arrest induction solution for use in the disclosed methods can comprise any of the cardiac arrest induction solutions disclosed herein including, but not limited to a cardiac arrest induction solution comprising a calcium channel blocker (such as, for example Magnesium including Magnesium in the form of Magnesium sulfate), a sodium channel blocker (such as, for example, Lidocaine), and a free radical scavenger (such as, for example, Mannitol).

The cardiac arrest induction solution can further comprise Potassium. In one aspect, Potassium is provided as a component of the cardiac arrest solution. However, it is contemplated herein that Potassium may also be provided as a separate component and mixed with the cardiac arrest induction solution prior to administration, administered concurrently with the cardiac arrest induction solution, or provided sequentially with the cardiac arrest induction solution. Because potassium can be provided separately or not at all in the disclosed methods, it is contemplated herein that the cardiac arrest induction solution for use in the disclosed methods may consist essentially of a calcium channel blocker (such as, for example Magnesium including Magnesium in the form of Magnesium sulfate), a sodium channel blocker (such as, for example, Lidocaine), and a free radical scavenger (such as, for example, Mannitol). In another aspect, it is contemplated herein that the cardiac arrest induction solution for use in the disclosed methods may consist essentially of Potassium, a calcium channel blocker (such as, for example Magnesium including Magnesium in the form of Magnesium sulfate), a sodium channel blocker (such as, for example, Lidocaine), and a free radical scavenger (such as, for example, Mannitol). In one aspect, the methods disclosed herein for maintaining a patient's heart during cardiac arrest include arresting the heart by perfusing the heart with any of the cardiac arrest induction solutions disclosed herein in combination with whole blood. For example, the cardiac arrest induction solution for the microplegia can comprise (a) from about 0.1 to about 25 milliequivalents of K+, (b) from 0.1 to about 200 milligrams Lidocaine, (c) from about 0.1 to about 2 grams Magnesium Sulfate, and (d) from about 0 to about 5 grams Mannitol per liter of carrier blood delivered.

It is understood and herein contemplated that the cardiac reanimation solution for use in the disclosed methods can comprise any of the cardiac reanimation solutions disclosed herein including, but not limited to a cardiac reanimation solution comprising a calcium channel blocker (such as, for example Magnesium including Magnesium in the form of Magnesium sulfate), a sodium channel blocker (such as, for example, Lidocaine), and a free radical scavenger (such as, for example, Mannitol) or a cardiac reanimation solution comprising a calcium channel blocker (such as, for example Magnesium including Magnesium in the form of Magnesium sulfate), a sodium channel blocker (such as, for example, Lidocaine), and beta receptor blocker (such as, for example, the cardioselective beta receptor blocker esmolol).

In a further aspect, the cardiac reanimation solution can comprise sodium bicarbonate. Thus, in one aspect disclosed herein are methods of preserving a heart wherein the cardiac reanimation solution comprises of a calcium channel blocker (such as, for example Magnesium including Magnesium in the form of Magnesium sulfate), a sodium channel blocker (such as, for example, Lidocaine), and a free radical scavenger (such as, for example, Mannitol) and Sodium Bicarbonate. Also disclosed herein are methods of preserving a heart wherein the cardiac reanimation solution comprises of a calcium channel blocker (such as, for example Magnesium including Magnesium in the form of Magnesium sulfate), a sodium channel blocker (such as, for example, Lidocaine), and beta receptor blocker (such as, for example, the cardioselective beta receptor blocker Esmolol), and Sodium Bicarbonate. In one aspect, the reanimation solution of the disclosed methods of preserving a heart comprises Magnesium sulfate, Mannitol, Lidocaine, and Sodium Bicarbonate. In another aspect, the reanimation solution of the disclosed methods of preserving a heart comprises Magnesium sulfate, Esmolol, Lidocaine, and Sodium Bicarbonate. In a further aspect, the reanimation solution of the disclosed methods of preserving a heart consists essentially of Magnesium sulfate, Mannitol, Lidocaine, and Sodium Bicarbonate. In a further aspect, the disclosed cardiac reanimation solution of the disclosed methods can further comprise a free radical scavenger (such as, for example, Mannitol) and a beta receptor blocker (such as, for example, the cardioselective beta receptor blocker Esmolol).

It is understood that the concentration of the components of the cardiac arrest induction solution and cardiac reanimation solution can be any combination of concentrations of the various components disclosed herein. It is further understood and contemplated herein that the cardiac arrest induction solution and the cardiac reanimation solution for use in the disclosed methods can comprise the same components (for example, Magnesium including Magnesium in the form of Magnesium sulfate; Lidocaine, and Mannitol). It is further understood and contemplated herein that the cardiac arrest induction solution and cardiac reanimation solution can not only comprise the same components but actually be the same solution. For example, in one aspect, disclosed herein are methods of preserving a heart during a surgical procedure comprising administering a cardiac arrest induction solution to the blood of a subject, perfusing the heart with the blood containing the cardiac arrest induction solution, and reanimating the heart with a cardiac reanimation solution, wherein the cardiac arrest induction solution and the cardiac reanimation solution both consist essentially of a calcium channel blocker (such as, for example Magnesium including Magnesium in the form of Magnesium sulfate), a sodium channel blocker (such as, for example, Lidocaine), and a free radical scavenger (such as, for example, Mannitol).

It is understood and herein contemplated that the disclosed cardiac arrest induction solution and cardiac reanimation solution can be administered intravenously through any means appropriate for induction of cardiac arrest and restoring cardiac function including but not limited to microplegia. As used herein, the expression “microplegia” refers to an all blood process whereby concentrated components may be delivered for paralysis of the heart. Accordingly, in one aspect, disclosed herein are methods of preserving a heart during a surgical procedure comprising administering a cardiac arrest induction solution to the blood of a subject (including but not limited to, for example the whole blood of a subject), perfusing the heart with the blood containing the cardiac arrest induction solution, and reanimating the heart with a cardiac reanimation solution; wherein the cardiac arrest induction solution and cardiac reanimation solution are administered via microplegia.

One of the advantages of the methods and compositions disclosed herein is the ability to induce prolonged cardiac arrest preserving heart function through a single dose of cardiac arrest induction solution. Accordingly, in one aspect, disclosed herein are methods of preserving a heart during a surgical procedure comprising administering a cardiac arrest induction solution to the blood of a subject, perfusing the heart with the blood containing the cardiac arrest induction solution, and reanimating the heart with a cardiac reanimation solution; wherein a single dose of the cardiac arrest induction solution is administered by microplegia.

The arrest induced by the disclosed methods can be sustained up to 6 hrs following administration of the cardiac arrest induction solution without the need for further administration of the cardiac arrest induction solution. In one aspect disclosed herein are methods of preserving a heart wherein the cardiac reanimation solution is administered 10 min, 20 min, 30 min, 40 min, 50 min, 1 hr, 2 hrs, 3 hrs, 4 hrs, 5 hrs, 6 hrs, 7 hrs, 8 hrs, 9 hrs, or 10 hrs or less following administration of cardiac arrest induction solution. For example, disclosed herein are methods wherein the cardiac reanimation solution is administered 1, 2, 3, 4, 5, or 6 hours or less following administration of the cardiac arrest induction solution.

It is understood and herein contemplated that the timing of the administration of the cardiac reanimation solution is typically referenced in the art based on the timing of removal of the cross clamp. Typically, the cardiac reanimation solution is administered 10, 11, 12, 13, 14, 15, 16 17, 18, 19, 20, 21, 22, 23, 24, 25, 26, 27, 28, 29, or 30 minutes prior to cross clamp removal.

It is further understood that situations can arise where a subsequent dose administration of the cardiac arrest solution is desired. Thus, in one aspect disclosed herein are methods of preserving a heart further comprising administering one or more subsequent dose of the cardiac arrest induction solution. Where more than one dose of the cardiac arrest induction solution is used, it is understood and herein contemplated that the cardiac reanimation solution can be administered 10 min, 20 min, 30 min, 40 min, 50 min, 1 hr, 2 hrs, 3 hrs, 4 hrs, 5 hrs, 6 hrs, 7 hrs, 8 hrs, 9 hrs, or 10 hrs or less following administration of any subsequent dose of cardiac arrest induction solution.

In one aspect the methods disclosed herein provide perfusing the heart with a cardiac arrest induction solution of about 10 to 20 milliliters per kilogram of delivery volume to achieve sufficient arrest. In one aspect may provide delivery of the cardiac arrest induction solution in subsequent doses of 100 to 500 milliliters about every 10 to 130 minutes during cardiac arrest as deemed necessary, but most often unneeded prior to 60 minutes in to an arrest period after induction.

Temperature of delivery of the cardiac arrest induction solution is factor the patient's response to the disclosed methods. In one aspect the disclosed methods can be used for preserving a patient's heart by perfusing the cardiac arrest induction solution disclosed herein in combination with the blood carrier having a temperature of about 5° to 37° C. The arresting dose or subsequent dose delivery temperature is about 5° to 20° C.

The temperature range for any reanimation dose can be about 30° to 37° C. The reanimation dose is associated with higher levels of coronary blood flow and may be given immediately preceding cross clamp removal with additional components such as esmolol, a beta blocker for heart rate control, and/or sodium bicarbonate for an additional buffering aspect with regards to extended ischemic time as deemed necessary. Reanimation occurs in a slow, controlled manner as blood flow is reestablished.

It will be generally understood that reestablishment of blood flow after the cross clamp is removed is what restarts the previously arrested heart, but function and timing of this reanimation may be improved by the aforementioned warm reanimation dose due to increased coronary blood flow and benefits of additives listed herein.

It will be appreciated by those skilled in the art that this process for preserving a patient's heart during microplegia includes delivering the solution of this invention in combination with whole blood directly into the aortic root, coronary ostia, retrograde via the coronary sinus, or via conduit (arterial or venous in nature). The most common route utilized in this invention is antegrade, via the aortic root, with little, if any, need for retrograde delivery.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Cardiac Arrest Induction Solution

To further detail the solution of this invention, an example is provided. Below is one composition of 32 milliliters of the solution carried by 1 liter of the patient's whole blood that may be given.

| | | |
|---|---|---|
| K⁺ | 22 | milliequivalents |
| Mg⁺⁺ | 1.6 | grams |
| Lidocaine | 104 | milligrams |
| Mannitol | 2.6 | grams |

### 2. Example 2

### Cardiac Reanimation Solution

This is an example of a warm reanimation dose that may be utilized in the period just prior to cross clamp removal. Here the composition consists of 22 milliliters of the solution carried by 450 milliliters of the patient's whole blood.

| | | |
|---|---|---|
| Lidocaine | 100 | milligrams |
| Magnesium Sulfate | 1 | gram |
| Sodium Bicarbonate | 5 | milliequivalents |
| Esmolol | 100 | milligrams |

What is claimed is:

1. A cardiac arrest induction solution consisting essentially of Potassium, Lidocaine, Magnesium, and Mannitol.

2. The cardiac arrest induction solution of claim 1, wherein the amount of Potassium comprises 0.1 to about 25 mmol per liter of blood cardioplegia delivered.

3. The cardiac arrest induction solution of claim 1, wherein the amount of Lidocaine comprises 0.1 to 200 mg per liter of blood cardioplegia delivered.

4. The cardiac arrest induction solution of claim 1, wherein the amount of Magnesium comprises 0.1 to 2 g per liter of blood cardioplegia delivered.

5. The cardiac arrest induction solution of claim 4, wherein the Magnesium is provided as Magnesium sulfate.

6. The cardiac arrest induction solution of claim 1, wherein the amount of Mannitol comprises 0.1 to 5 g per liter of blood cardioplegia delivered.

7. The cardiac arrest induction solution of claim 1, wherein the carrier solution is a bufferless solution.

8. The cardiac arrest induction solution of claim 7, wherein the solution does not comprise NaHCO₃.

9. A cardiac arrest induction solution consisting essentially of Lidocaine, Magnesium, and Mannitol.

10. The cardiac arrest induction solution of claim 9, wherein the amount of Lidocaine comprises 0.1 to 200 mg per liter of blood cardioplegia delivered.

11. The cardiac arrest induction solution of claim 9, wherein the amount of Magnesium comprises 0.1 to 2 g per liter of blood cardioplegia delivered.

12. The cardiac arrest induction solution of claim 11, wherein the Magnesium is provided as Magnesium sulfate.

13. The cardiac arrest induction solution of claim 9, wherein the amount of Mannitol comprises 0.1 to 5 g per liter of blood cardioplegia delivered.

14. The cardiac arrest induction solution of claim 9, wherein the solution is a bufferless solution.

15. The cardiac arrest induction solution of claim 14, wherein the carrier solution does not comprise Sodium Bicarbonate.

16. A cardiac reanimation solution comprising Lidocaine, Magnesium, Sodium Bicarbonate, and Esmolol.

17. The cardiac reanimation solution of claim 16, wherein the amount of Lidocaine comprises 0.1 to 200 mg per liter of blood cardioplegia delivered.

18. The cardiac reanimation solution of claim 16, wherein the amount of Magnesium comprises 0.1 to 2 g per liter of blood cardioplegia delivered.

19. The cardiac reanimation solution of claim 18, wherein the Magnesium is provided as Magnesium sulfate.

20. The cardiac reanimation solution of claim 16, wherein the amount of Esmolol comprises 0 to 200 mg per liter of blood cardioplegia delivered.

21. The cardiac reanimation solution of claim 16, further comprising Sodium Bicarbonate.

22. The cardiac reanimation solution of claim 21, wherein the amount of Sodium Bicarbonate comprises 0.1 to 25 mmol per liter of blood cardioplegia delivered.

23. A method of preserving a heart during a surgical procedure comprising administering a cardiac arrest induction solution to the blood of a subject, perfusing the heart with the blood containing the cardiac arrest induction solution, and reanimating the heart with a cardiac reanimation solution.

24. The method of claim 23, wherein the cardiac arrest induction solution consists essentially of Magnesium, Lidocaine, and Mannitol.

25. The method of claim 24, wherein the method further comprises administering Potassium to the blood.

26. The method of claim 25, wherein the Potassium and the cardiac arrest solution are administered concurrently.

27. The method of claim 25, wherein the Potassium and the cardiac arrest solution are administered sequentially.

28. The method of claim 25, wherein the Potassium and the cardiac arrest solution are combined prior to administration.

29. The method of claim 23, wherein the cardiac arrest induction solution consists essentially of Potassium, Magnesium, Lidocaine, and Mannitol.

30. The method of claim 23, wherein the cardiac arrest induction solution is administered to the blood via a microplegia device.

31. The method of claim 23, wherein only a single dose of the cardiac arrest induction solution is administered.

32. The method of claim 23, further comprising administering a subsequent dose of the cardiac arrest induction solution.

33. The method of claim 23, wherein the cardiac reanimation solution is administered 3 hrs or less following administration of the cardiac arrest induction solution.

34. The method of claim 23, wherein the cardiac reanimation solution consists essentially of Lidocaine, Magnesium, Sodium Bicarbonate, and Esmolol.

35. The method of claim 23, wherein the cardiac reanimation solution is administered to the blood via a microplegia device.

36. The method of claim 23, wherein the delivery temperature is from about 6 degrees Celsius to about 37 degrees Celsius.

37. A method of preserving a heart during a surgical procedure comprising administering a cardiac arrest induction solution to the blood of a subject, and perfusing the heart with the blood containing the cardiac arrest induction solution.
